(19) United States
(12) Patent Application Publication
(10) Pub. No.: US 2026/0297039 A1
(43) Pub. Date: Oct. 1, 2026

(54) MELAMINE PROCESS WITH UREA MELT-BASED WASHING OF MELAMINE OFFGAS

(71) Applicant: Casale SA, Lugano (CH)

(72) Inventors: Gabriele Di Carlo, Lugano (IT); Maurizio Cipolla, Lecco (IT); Simone Gamba, Pagazzano (BG) (IT)

(73) Assignee: Casale SA, Lugano (CH)

(21) Appl. No.: 19/479,681

(22) PCT Filed: May 17, 2024
(86) PCT No.: PCT/EP2024/063723

§ 371 Date: Oct. 28, 2025

(30) Foreign Application Priority Data
May 25, 2023 (EP) 23175262.7

(51) Int. Cl. C07D 251/60; B01D 53/14; F04D 1/00; F04D 29/08

(52) U.S. Cl. CPC C07D 251/60; B01D 53/1412; B01D 53/1475; B01D 53/1493; F04D 1/00; F04D 29/086; B01D 2257/406; B01D 2257/504

(57) ABSTRACT

A process for the synthesis of melamine from urea wherein offgas from the melamine synthesis reaction is washed in a scrubber with urea melt, the urea melt removed from the scrubber is sent to a urea melt pump and partly recirculated to the scrubber, said urea melt pump includes a dual seal pressurized with water.

### FIELD OF APPLICATION

The invention is in the field of industrial production of melamine. The invention particularly pertains to a high-pressure melamine synthesis process including washing of the melamine offgas with urea melt.

### PRIOR ART

The technology for production of melamine at an industrial scale includes the non-catalytic high-pressure (HP) process and the low-pressure (LP) catalytic process. The non-catalytic high-pressure process is considered the most advantageous and is becoming predominant.

In the high-pressure process, a urea melt is reacted at pressure which is generally above 70 bar, typically 75 to 200 bar. The temperature of reaction is typically around 375° C.

The melamine-containing product stream is sent to further treatments, typically performed at a lower pressure. Said treatments may include quenching, purification, crystallization, solid-liquid separation and drying, to obtain solid melamine of a desired purity. Typically, melamine purification and crystallization are carried out in an alkaline environment and ammonia or sodium hydroxide are the most commonly used alkaline agents.

The conversion of urea to melamine produces a gaseous stream (“melamine offgas”) containing predominantly ammonia and carbon dioxide, some melamine, and other minor components.

It is generally desired to remove melamine from the offgas, to recover melamine and to purify the offgas for a further use, such as recycling the offgas as a feed material for a tied-in urea plant. A known technique to purify the melamine offgas is washing with urea melt in a suitable scrubber. A portion of the available urea melt feed can be used to this purpose, and the urea melt after washing can be sent to the melamine synthesis section. U.S. Pat. No. 7,311,759 discloses for example a double-stage scrubbing process of the melamine offgas.

The urea melt collected from the scrubber is sent to a urea melt pump. Part or all of the urea melt delivered by the pump is sent to the melamine reactor; in some embodiments part is sent to the melamine reactor and part is recirculated to the scrubber via an external cooler.

In the washing process, the contact between the offgas and the urea melt generates melamine precursors such as ammeline and cyanuric acid. Therefore, the urea melt removed from the scrubber contains dissolved gas (predominantly ammonia) and melamine precursors.

A technical challenge in connection with said urea melt pump (receiving the urea melt from the scrubber) is to avoid an escape of the urea melt, which would cause undesired emission of ammonia and possible damages to the equipment due to the urea melt which easily crystallizes if cooled. Accordingly, the urea melt pump must be equipped with a suitable seal. The prior art suggests a canned motor pump which is inherently leak-free as the motor and the pump are integrated in the same structure and the liquid remains sealed within the structure.

However, the canned pump has disadvantages. The lubrication of a canned motor pump is performed by the process fluid, which is forced to pass through small orifices. In the present case, the process fluid is the urea melt which may contain solid products formed in the scrubber, especially if the scrubber reaches a temperature higher than expected. It is known that above 250° C. thermal decomposition of urea occurs quickly with formation and precipitation of products such as cyanuric acid, ammelide, ammeline. Solid particles contained in the process fluid may damage the pump and cause a shut-down of the entire plant.

During a stop, for example for maintenance, the pump must be thoroughly cleaned, otherwise products from crystallization of the process fluid may remain trapped in the pump, particularly between the rotor and the stator, possibly damaging the pump when restarted.

### SUMMARY OF THE INVENTION

The invention aims to overcome the above drawbacks of the prior art. The aim is reached with a process according to the claims.

A urea melt stream removed from the scrubber is sent to a urea melt pump with a water-pressurized dual seal arrangement. Said dual seal arrangement uses water under pressure (“sealing water”) as a barrier fluid. According to the invention, the flow rate of sealing water entering the process side of the pump through the double seal is not greater than a maximum value, which is 100 kg/h or more preferably 50 kg/h, or 10 kg/h, or 5 kg/h, or even more preferably 1 kg/h.

The invention goes against a prejudice in the prior art which discourages the use of a dual seal, with water as barrier fluid, for the pump of the urea melt removed from the scrubber. Said sealing technique was considered unsuitable for this specific application because water entering the process side can react with urea, forming gaseous ammonia and carbon dioxide. As a matter of fact, the reaction of urea and water reduces the urea available for the synthesis of melamine and gaseous products may cause cavitation; however, the invention is based on the judicious hindsight that a sufficiently small amount of water renders the above drawbacks tolerable or even negligible, whereas the dual seal eliminates the disadvantages of the canned motor pump.

### DESCRIPTION OF THE INVENTION

In a broad aspect, the invention relates to a process for the synthesis of melamine including the steps of:
reacting a feed stream of urea melt in a melamine synthesis section, according to non-catalytic high-pressure synthesis conditions, to generate a raw melamine product and an offgas comprising ammonia and carbon dioxide,
washing said offgas with urea melt in a scrubber,
wherein a urea melt stream removed from the scrubber is sent to a urea melt pump, and at least part of the urea melt delivered by said pump is sent to the melamine synthesis section as feed for the synthesis of melamine,
wherein said urea melt pump includes a pressurized dual seal for sealing a process side of the pump traversed by the urea melt, said dual seal being pressurized with sealing water under pressure,
wherein an amount of sealing water enters the process side of the pump through said dual seal, thus mixing with the urea melt processed by the pump,
said amount being not greater than 100 kg/h.

The term dual seal is understood in accordance with UNI-EN ISO 21049:2004 or in accordance with API standard 682, fourth edition, May 2014. It denotes that the pump has two mechanical seals with a barrier fluid between the seals. In the present invention, said barrier fluid is water at a suitable pressure.

The urea melt pump is typically a centrifugal pump.

The term sealing water denotes the water used as barrier fluid in the pump. The sealing water is not necessarily fresh water or pure water. The barrier fluid can be pure water (e.g., boiler feed water or steam condensate) or a water stream coming from the melamine plant. Said water stream coming from the melamine plant can contain dissolved NH3 and CO2, and traces of melamine and OATs. Said water stream has preferably a water content of at least 96% by mass, more preferably of at least 98% by mass and even more preferably of at least 99% by mass. Said stream is still named “water” for simplicity, although it contains contaminants.

As stated above, the amount of sealing water entering the pressure side of the pump is, most preferably, not greater than 1 kg/h.

The suction pressure of said pump is preferably 50 bar to 200 bar. The head of said pump is preferably 10 to 250 meters of liquid column (MLC).

In a preferred embodiment, the volumetric rate of urea melt delivered by said pump over the capacity of the process is 3 to 30 m³ of urea melt per ton of solid melamine obtained in the process. If urea melt delivered by the pump is sent entirely to the melamine synthesis section, said volumetric rate is preferably 3 to 11 m³ per ton of melamine. If otherwise, a portion of the urea melt from the pump is recirculated to the scrubber, said rate is preferably 11 to 30.

The symbol ton denotes 1000 kg. The solid melamine is understood as the solid melamine obtained from processing the melamine-containing synthesis effluent (raw melamine melt). The treatment of the raw melamine melt is performed at a pressure lower than the synthesis pressure and may include quenching, purification, crystallization, separation of the so obtained crystals of melamine from the remaining liquor and drying of said crystals. The mass rate of said solid melamine product normally denotes the capacity of the plant.

In a preferred embodiment, the capacity of the process is 5 to 15, preferably 5 to 10 tons of melamine per hour, said capacity being the amount solid melamine that is obtained from the process.

In a preferred embodiment, some of the urea melt delivered by the urea melt pump is recirculated to the same scrubber via a recirculation line. The remaining portion of urea melt delivered by the pump is sent to the melamine synthesis section. The ratio between the urea melt delivered by said pump and the solid melamine product of the melamine plant is preferably between 11 and 30 m³ of urea melt per ton of melamine.

The urea melt which is delivered by said urea pump and recirculated to the scrubber is preferably cooled in a urea melt cooler before introduction into the scrubber. More preferably, it is cooled to a temperature of at least 165° C. and more preferably in the range 165 to 245° C.

Said urea melt cooler is preferably a shell-and-tube apparatus. The above temperature is preferably the temperature of the urea melt at the outlet of said apparatus. Preferably the recirculated urea melt is cooled in the tube side and heat removed from the urea melt is used to produce steam in the shell side of said urea melt cooler. The temperature of the steam produced in the shell side is preferably between 160° C. and 240° C.

The selected temperature of at least 165° C., preferably in the range 165° C. to 245° C., to which the recirculated urea melt is cooled prior to reintroduction in the scrubber, prevents the precipitation of melamine cyanurate and corrosion issues. A temperature of the urea melt above 165° C. avoids the precipitation of the cyanurate, whereas a temperature not greater than 245° C. is beneficial to avoid corrosion, i.e. to control the corrosive effect of the urea melt flowing through the urea melt cooler.

The non-recirculated portion of the urea melt can be sent to the melamine synthesis section. Accordingly, a first portion of the urea melt delivered by said pump is recirculated to the scrubber and a second portion of the urea melt delivered by said pump is sent to the melamine synthesis section. Said second portion is preferably the remaining portion of the urea melt stream leaving the pump.

Said scrubber may be single-stage or may include two stages. The contact between the melamine offgas and the urea melt is preferably counter-current. Preferably, the scrubber is arranged vertically.

In an embodiment, the cooling of urea melt is performed internally in the scrubber. Accordingly, a urea melt stream collected from bottom of the scrubber is sent to the urea melt pump and the urea melt delivered by said pump can be sent entirely to the melamine synthesis section. In an embodiment, the scrubber may have an internal recirculation promoted by gravity.

In a preferred embodiment, the scrubber includes a first stage and a second stage, in the first stage the melamine offgas is contacted in counter-current direction with the urea melt comprising the recirculated urea melt loaded with ammonia and melamine precursors; in the second stage the offgas emerging from the first stage is contacted in counter-current with a fresh urea melt, said fresh urea melt being introduced in the second stage and traversing in sequence the second stage and the first stage. The two stages operate in sequence, so that the partially purified offgas effluent from the first stage is processed in the second stage. The first stage can be operated at a higher temperature than the second purification step, or the two steps may be performed substantially at the same temperature.

A preferred embodiment of a two-stage scrubber is as follows. The second purification stage is placed above the first purification stage; the melamine offgas flows upward in the first stage and then in the second stage; a urea melt is sprayed over the offgas from top of the second stage and flows downward through the second stage and the first stage; a urea melt loaded with ammonia and melamine precursors is collected at the bottom of the first stage and a portion thereof is recirculated to the same first stage after cooling. The melamine offgas is scrubbed in counter-current firstly with the urea melt from the second stage and the urea melt recirculated in the first stage; then with the fresh urea melt introduced in the second stage. Preferably the first stage operates in a temperature range of 170° C. to 250° C., whereas the second stage operates at 135° C. to 230° C.

In some embodiments, the offgas washing includes the addition of carbon dioxide. A carbon dioxide stream can be added to the melamine offgas before it enters the scrubber, or can be introduced separately into the scrubber. In an embodiment with two stages, addition of carbon dioxide is made preferably to the first stage.

The washing of the melamine offgas with urea melt is performed at a high pressure, preferably of at least 50 bar and more preferably equal to or substantially equal to the melamine synthesis pressure. The offgas washing may be performed at a pressure slightly less than the melamine synthesis pressure, wherein the difference is not more than 20 bar or not more than 5 bar. The purified offgas after washing may be recycled to a tied-in urea plant.

According to an embodiment, the off-gas is introduced in the scrubber via an off-gas distributor above or below a liquid level of the urea melt containing ammonia and melamine precursors. A preferred embodiment of said offgas distributor is disclosed in U.S. Pat. No. 7,311,759. The urea melt may be introduced in the scrubber as a single stream or divided into a plurality of streams and introduced at multiple locations of the scrubber, e.g. at different vertical elevation.

The urea melt withdrawn from the scrubber (be it a single stage or two-stage) can be a mixture of fresh urea melt and recirculated urea melt. The stream of urea melt delivered by the pump to the melamine synthesis section may be added with fresh urea melt to form the feed of the melamine synthesis section.

The fresh urea melt is preferably a urea melt obtained from a urea plant after recovery of unreacted matter and evaporation of water. Typically, the urea melt contains at least 96% urea, the balance being residual water and unavoidable impurities.

In the melamine synthesis section, the urea melt is reacted under non-catalytic high pressure melamine synthesis conditions to generate a raw melamine product and a stream of melamine offgas comprising ammonia, carbon dioxide, melamine and minor components. The melamine synthesis pressure is preferably 70 bar or above, for example 70 bar to 200 bar.

According to a preferred embodiment, the synthesis of melamine includes a conversion step and a stripping step, wherein said conversion step includes reacting said urea melt feed stream under suitable melamine synthesis conditions to generate a raw melamine product, and said stripping step includes the stripping of said raw melamine product in the presence of gaseous ammonia, to remove carbon dioxide contained in the raw melamine.

In some embodiments, the melamine synthesis section includes a single reactor, from which the raw melamine and the melamine offgas are withdrawn. In other embodiments, said melamine synthesis section includes a primary reactor where urea melt is reacted, followed by a secondary reactor where the melamine-containing effluent of the primary reactor is stripped with gaseous ammonia. In such embodiments, each of the primary reactor and the secondary reactor produce a respective stream of melamine offgas. Both melamine offgas streams are made predominantly of ammonia and carbon dioxide, although they may differ in composition.

The melamine offgas subject to scrubbing with urea melt, in accordance with embodiments the invention, may include only the melamine offgas stream from the primary reactor or both melamine offgas streams from the primary reactor and secondary reactor, possibly combined into a single stream. In a further embodiment, a combined reactor performs the function of the primary reactor and secondary reactor; to this purpose, said combined reactor includes a primary reaction stage and a secondary reaction stage.

In a combined urea-melamine embodiment, ammonia and carbon dioxide are reacted to form a urea solution in a urea synthesis section, the urea solution is processed in at least one recovery section to obtain a purified urea solution and water is removed from the solution to form a urea melt. Said urea melt is used in the above-described process for synthesis of melamine. The melamine offgas generated during the synthesis of melamine is recycled to the production of urea.

A further aspect of the invention is a scrubbing section according to the claims.

Said feeding means of the water under pressure (barrier fluid of the seal) may include a suitable pump, reservoir and piping.

The present invention goes against the consistent teaching in the prior art to use a canned pump which prevents any introduction of water in the urea melt, following the conventional wisdom that injection of water may induce corrosion and/or cavitation of the pump (due to formation of gaseous ammonia and CO2) and affect the yield of the process. The invention discloses injection of a small but non null amount of water in a pressurize dual seal of a urea melt pump for the specific application of recirculation of urea melt in a melamine offgas scrubbing process, which is not suggested in the prior art; the invention further discloses that such injection of water provides the desired seal of the urea melt pump without the drawbacks feared in the prior art.

### EXAMPLE

It is assumed that 100 kg/h of water enter the process side of the pump and are entirely mixed with the urea melt processed by the pump. Said amount of 100 kg/h of water would hydrolize 333 kg/h of urea according to the reaction:

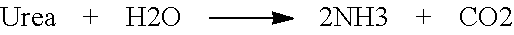

In a melamine plant of a standard industrial size, the consumption of urea is typically 3.1 t_{UREA}/t_{MEL} (tons of urea per ton of melamine produced). Assuming a capacity of 5 t/h (tons per hour) of melamine, the above loss of 333 kg/h of urea would correspond, in a worst-case scenario, to 0.333/5=0.067 t_{UREA}/t_{MEL} which means the specific consumption is increased only by 2.1%. With an injection of 10 kg/h of water, this figure is reduced to 0.21%. For even smaller amounts of water, the increase of specific consumption reduces correspondingly.

### DESCRIPTION OF THE FIGURES

FIG. 1 is a scheme of a process according to a first embodiment of the invention.

FIG. 2 is a scheme of a second embodiment of the invention.

FIG. 3 is a scheme of a third embodiment.

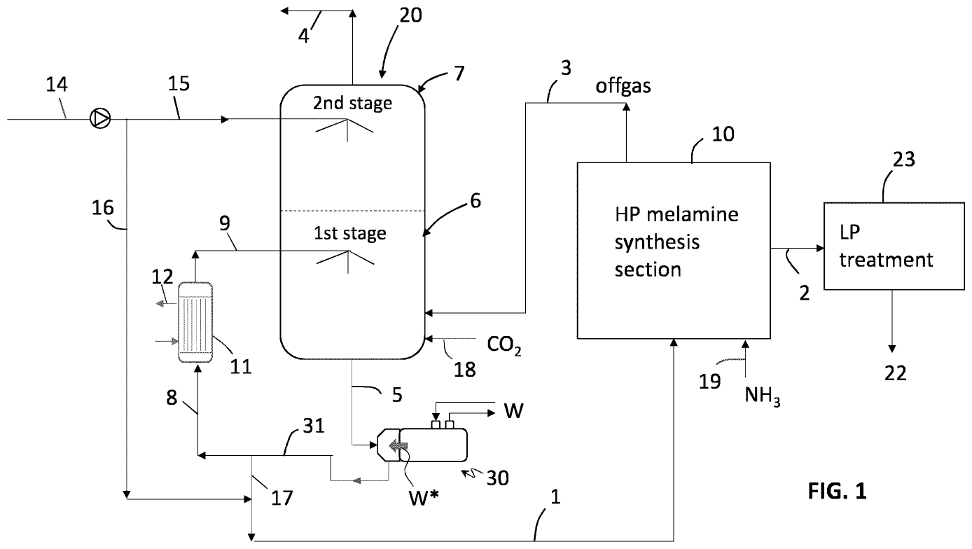

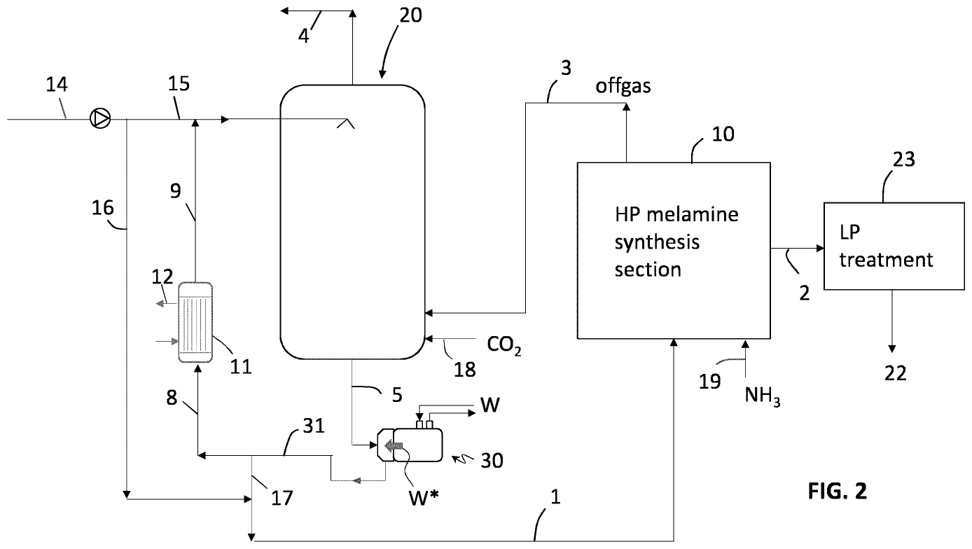

The main items in FIGS. 1-2 are:
10 melamine synthesis section
1 urea melt feed of the melamine section 10
2 raw melamine melt effluent from the melamine section 10
3 offgas withdrawn from the melamine section 10 (“melamine offgas”)
23 low-pressure (LP) treatment section of the raw melamine melt 2
20 scrubber for washing the melamine offgas 3
14 fresh urea melt from a urea plant
15 fresh urea melt input to the scrubber 20
16 fresh urea melt directed to the melamine synthesis section 10
5 urea melt collected from the scrubber 20 (after the scrubbing process)
30 urea melt recirculation pump
31 urea melt delivered by the pump 30
8 urea melt recirculated to the scrubber 20
17 urea melt sent to the synthesis section 10
11 urea melt cooler
4 purified offgas collected from the scrubber 20

The high-pressure melamine synthesis section 10 is supplied with the urea melt feed stream 1 and gaseous ammonia 19. Ammonia 19 is injected in the synthesis section 10 to act as a stripping agent to remove carbon dioxide from the raw melamine. The melamine synthesis reaction produces the stream 2 of raw melamine and the melamine offgas 3. Said melamine offgas 3 contains carbon dioxide, ammonia, some residual melamine and other minor components.

The synthesis section 10 may comprise two separate reactors wherein in the first reactor raw melamine is synthesized and in the second reactor the ammonia 19 is used as a stripping agent to remove the carbon dioxide from the raw melamine. Alternatively, the synthesis of raw melamine and stripping with ammonia can be carried out in a single reactor. In a preferred embodiment, a single reactor has coaxial zones for synthesis and stripping. For example, the synthesis is carried out in a central zone of the reactor and stripping is carried out in an annular zone wrapped around said central zone.

The raw melamine melt 2 is processed in the low-pressure section 23 to obtain solid melamine 22 of a desired purity. Said section 23 preferably includes quenching, purification, crystallization, solid-liquid separation and drying.

The scrubber 20 comprises a first stage 6 and a second stage 7. The second stage 7 is above the first stage 6. In the example, the scrubber 20 is traversed in counter-current by the melamine offgas flowing upward and by the urea melt flowing downward. The fresh urea melt 15 is sprayed in the second stage 7 and a recirculated urea melt in line 9 is sprayed in the first stage 6. The first stage 6 receives also a stream of gaseous carbon dioxide 18. The melamine offgas 3 enters the first stage 6 and flows upward traversing the first stage and the second stage 7, contacting the fresh and recirculated urea melt. The carbon dioxide 18 promotes the formation of the melamine precursors contained in the urea melt 5.

Effluents of the scrubber 20 are the purified offgas 4 and the urea melt 5 which, as a result of the washing process, contains ammonia and melamine precursors. The urea melt 5 is sent to the urea melt pump 30. Part of the urea melt stream 31 delivered by said pump 30 is recirculated to the scrubber 20 via lines 8 and 9. A remaining part is joined with the fresh urea melt stream 16 to form the urea melt feed 1.

The recirculated urea melt is cooled in the tube side of the urea melt cooler 11, which is a shell-and-tube heat exchanger. The cooled urea melt in line 9 leaves the cooler 11 at a temperature in the range 165° C. to 245° C. The shell side of the cooler 11 produces steam 12, preferably having a temperature of 160° C. to 240° C. Particularly preferably, said steam is saturated steam at 6 barg.

The urea melt pump 30 is a centrifugal pump with a pressurized dual seal. The barrier fluid of the pressurized seal is water W. Said water W has a suitable pressure to prevent leakage of the urea melt. Said pressure is greater than the suction pressure of the pump 30 and preferably greater than the pressure of the urea melt at line 31.

The pump 30 has a process side traversed by the urea melt 5 where energy is transferred to the urea melt 5 to raise its pressure. Said process side is separated from the outside (commonly termed “atmospheric side”) by the pressurized dual seal. Said pressurized dual seal includes a primary seal facing the process side and a secondary seal. A chamber between the primary seal and the secondary seal is filled with the sealing water W under pressure.

In operation, an amount of the sealing water, denoted by W*, enters the process side of the pump passing through the primary seal, due to unavoidable clearance (e.g. between a rotating part and a static part of the pump). Said water W* therefore mixes with the urea stream and enters the synthesis process with the stream 31. Said water W* however is not more than 100 kg/h and more preferred values are in accordance with the claims.

The purified offgas 4 emerging from the second stage 7 can be recycled to a urea plant not shown in the figure, for example to the urea plant which produces the urea melt 14.

FIG. 2 illustrates an embodiment where the scrubber 20 has a single stage. The recirculated urea melt, before or after cooling, is mixed with the fresh urea melt 15 and sprayed from top of the scrubber 20. FIG. 2 illustrates that the fresh urea melt 15 mixes with the recirculated urea melt in line 9 after cooling; in a variant (not shown), the fresh urea melt 15 mixes with urea melt in line 8 before cooling.

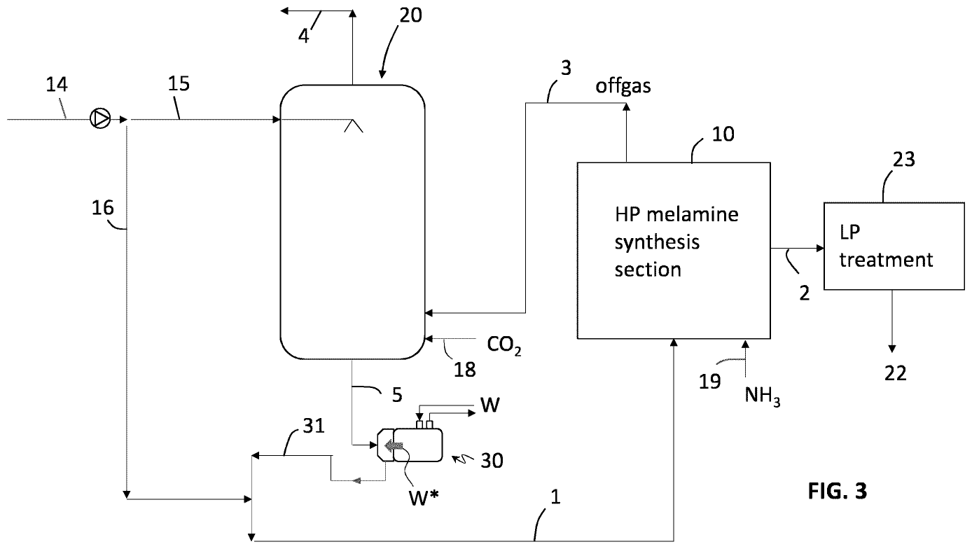

FIG. 3 illustrates an embodiment where the recirculation of the urea melt is made internally in the scrubber 20. A urea melt cooler can be installed internally in the scrubber to cool the recirculated urea melt. The urea melt stream 31 delivered by the pump 30 is sent entirely to the melamine section 10 after mixing with the fresh stream 16.

1. A process for the synthesis of melamine including the steps of:
reacting a feed stream of urea melt in a melamine synthesis section, according to non-catalytic high-pressure synthesis conditions, to generate a raw melamine product and an offgas comprising ammonia and carbon dioxide,
washing said offgas with urea melt in a scrubber,
wherein a urea melt stream removed from the scrubber is sent to a urea melt pump, and at least part of the urea melt delivered by said pump is sent to the melamine synthesis section as feed for the synthesis of melamine,
wherein said urea melt pump includes a pressurized dual seal for sealing a process side of the pump traversed by the urea melt, said dual seal being pressurized with sealing water (W) under pressure,
wherein an amount (W*) of sealing water enters the process side of the pump through said dual seal, thus mixing with the urea melt processed by the pump,
said amount (W*) being not greater than 100 kg/h.

2. The process according to claim 1, wherein said amount of sealing water entering the process side is not greater than 50 kg/h.

3. The process according to claim 1, wherein the suction pressure of said pump is 50 bar to 200 bar.

4. The process according to claim 1, wherein the head of said pump is 10 to 250 meters of liquid column.

5. The process according to claim 1, wherein the volumetric rate of urea melt delivered by said pump over the capacity of the process is 3 to 30 m3 of urea melt per ton of solid melamine obtained in the process.

6. The process according to claim 1, wherein the capacity of the process is 5 to 15 tons of melamine per hour, the capacity being the amount solid melamine that is obtained from the process.

7. The process according to claim 1, wherein: a first portion of the urea melt delivered by said pump is recirculated to the scrubber; a second portion of the urea melt delivered by said pump is sent to the melamine synthesis section.

8. The process according to claim 7, wherein said first portion of urea melt, which is recirculated to the scrubber, is cooled before introduction into the scrubber to a temperature in any one of the following ranges: 165° C. to 245° C., 170° C. to 235° C., 175° C. to 225° C. or 180° C. to 220° C.

9. The process according to claim 8, wherein said scrubber includes a first stage and a second stage, in the first stage the melamine offgas is contacted in counter-current direction with urea melt from the second stage and with the recirculated urea melt; in the second stage the offgas emerging from the first stage is contacted in counter-current with a fresh urea melt.

10. The process according to claim 1, said sealing water including fresh water and/or a water stream containing dissolved NH3 and CO2, and possibly traces of melamine and OATs, having a water content of at least 96% by mass.

11. A scrubbing section for melamine offgas withdrawn from a melamine synthesis section, said scrubbing section including:
a scrubber arranged to wash the melamine offgas with urea melt;
a urea melt pump arranged to receive a urea melt stream collected from said scrubber;
a line arranged to transport at least part of the urea melt delivered by said pump to the melamine synthesis section,
wherein said urea melt pump includes a water-pressurized dual seal, which is fed with sealing water (W) under a suitable pressure acting as barrier fluid in the seal, wherein said seal separates a process side of the pump from the outside environment,
said dual seal is configured so that an amount (W*) of sealing water entering the process side through said dual seal is not greater than 100 kg/h.

12. The scrubbing section according to claim 11, wherein said flow rate of sealing water entering the process side is not greater than 50 kg/h.

13. The scrubbing section according to claim 11, further including a line arranged to recirculate a portion of the urea melt delivered by said pump into the scrubber, said line including a urea melt cooler.

14. A process according to claim 2, wherein said amount of sealing water entering the process side is not greater than 10 kg/h.

15. A process according to claim 2, wherein said amount of sealing water entering the process side is not greater than 5 kg/h.

16. A process according to claim 2, wherein said amount of sealing water entering the process side is not greater 1 kg/h.

17. A process according to claim 10, said sealing water including fresh water and/or a water stream containing dissolved NH3 and CO2, and possibly traces of melamine and OATs, having a water content of at least 98% by mass.

18. A process according to claim 10, said sealing water including fresh water and/or a water stream containing dissolved NH3 and CO2, and possibly traces of melamine and OATs, having a water content of at least 99% by mass.

19. The scrubbing section according to claim 12, wherein said flow rate of sealing water entering the process side is not greater than 10 kg/h.

20. The scrubbing section according to claim 12, wherein said flow rate of sealing water entering the process side is not greater than 5 kg/h.

21. The scrubbing section according to claim 12, wherein said flow rate of sealing water entering the process side is not greater than 1 kg/h.
